(19) United States
(12) Patent Application Publication
(10) Pub. No.: US 2025/0194952 A1
(43) Pub. Date: Jun. 19, 2025

(54) NON-CONTACT CARDIOGRAM MEASUREMENT METHOD

(71) Applicant: ZHEJIANG UNIVERSITY, Hangzhou (CN)

(72) Inventors: Shuqin DONG, Hangzhou (CN); Lixin RAN, Hangzhou (CN)

(73) Assignee: ZHEJIANG UNIVERSITY, Hangzhou (CN)

(21) Appl. No.: 17/766,242

(22) PCT Filed: May 6, 2020
(86) PCT No.: PCT/CN2020/088812

§ 371 Date: Apr. 2, 2022

(30) Foreign Application Priority Data
May 6, 2019 (CN) 201910371734.3

(51) Int. Cl. A61B 5/11; A61B 5/00; A61B 5/02

(52) U.S. Cl. CPC A61B 5/1102; A61B 5/02028; A61B 5/1126; A61B 5/7239

(57) ABSTRACT

A non-contact cardiogram measurement method is disclosed. The method includes: measuring, by a Doppler radar or a pulse radar, a distance change between the radar and a skin surface through an electromagnetic wave, a light wave and a sound wave; deriving a displacement curve indicating a change in a human heart volume; taking first-order and second-order derivations of the displacement curve to acquire velocity and acceleration curves; taking zero points and extreme points of the velocity and acceleration curves as velocity and acceleration information, and as characteristic points of different stages of atrial and ventricular systole and diastole; acquiring time, volume change, velocity and acceleration information of atrial and ventricular systole and diastole in a cardiac cycle. The present disclosure features low cost and low radiation hazard, avoids direct contact with the human body, and provides a convenient and easy way for daily cardiac diagnostic measurement of special patients.

### CROSS REFERENCE TO THE RELATED APPLICATIONS

This application is the national phase entry of International Application No. PCT/CN2020/088812, filed on May 6, 2020, which is based upon and claims priority to Chinese Patent Application No. 201910371734.3, filed on May 6, 2019, the entire contents of which are incorporated herein by reference.

### TECHNICAL FIELD

The present disclosure relates to a non-contact cardiogram measurement method, and in particular to a radar system with high linearity and sensitivity and a non-contact measurement method for a cardiogram of a human body based on the radar system.

### BACKGROUND

In modern society, the mortality rate of cardiovascular disease has always been the first among various diseases. Measurement and long-term monitoring of cardiograms are essential in the diagnosis and prevention of cardiovascular disease. Various cardiographs are used to measure cardiograms based on different principles and techniques. Currently, commercial echocardiographs are capable of measuring electrocardiograms (ECGs), ultrasonic cardiograms (UCG), phonocardiograms (PCGs), impedance cardiogram (ICGs), ballistocardiograms (BCGs) and magnetic resonance imaging (MRI)-based real-time ECGs. The measurement of cardiograms is essential for the diagnosis and daily care of cardiovascular disease.

Specialized cardiographs, such as the most commonly used electrocardiographs, rely on contact sensors. With the exception of the MRI-based ECGs, which are expensive and unsuitable for daily care, all other cardiograms are measured using contact sensors, such as ECG electrodes and UCG probes. However, contact measurements are cumbersome and unsuitable for premature infants and burn patients.

Cardiograms are graphs of basic cardiac activities measured by sensor devices over time. There are two forms of basic cardiac activities: electrical activity and mechanical activity. These two activities interact and correlate to push blood through the atria and ventricles into the lungs or the circulatory system, providing adequate blood flow to organs and tissues to supply oxygen and various nutrients, and to carry away the end products of metabolism.

### SUMMARY

In order to solve the problems existing in the prior art, the present disclosure proposes a non-contact cardiogram measurement method. The present disclosure extracts relevant characteristic points from a motion curve of a human thoracic surface to acquire key information of atrial and ventricular motion, and acquires a correlation between a bioelectrical signal that controls the diastole and systole of a human heart and changes in atrial and ventricular volumes that occur at the skin surface.

The present disclosure adopts the following specific technical solution:

The present disclosure measures, in a non-contact manner, slight displacements of thoracic and dorsal skin surfaces induced by changes in atrial and ventricular volumes during combined atrial and ventricular motion, and acquires a cardiogram depicting the atrial and ventricular motion in a cardiac cycle.

The method includes: measuring, by a Doppler radar or a pulse radar, a distance change between the radar and a skin surface through an electromagnetic wave, a light wave and a sound wave; deriving a displacement curve indicating a change in a human heart volume; processing the displacement curve to acquire time, velocity and acceleration information depicting a cardiogram; taking first-order and second-order derivations of the displacement curve (or performing other equivalent operation) to acquire velocity and acceleration curves; taking zero points and extreme points of the velocity and acceleration curves as velocity and acceleration information, and as characteristic points of different stages of atrial and ventricular systole and diastole, which are located on the displacement curve; and acquiring time, volume change, velocity and acceleration information of atrial and ventricular systole and diastole in a cardiac cycle.

The measurement is performed from a back of a subject, and a radar sensor is placed facing a dorsal skin of the subject.

The radar sensor is used to measure a skin surface displacement, and a direct current (DC) offset compensation is performed during a measurement process to improve the measurement accuracy, which comprises locating a center of a circle of a baseband signal constellation diagram based on a baseband signal acquired by quadrature down-conversion of a radar receiver in the radar sensor, determining a DC offset component by a center coordinate, and correcting and compensating the baseband signal by the DC offset component.

The radar sensor includes a radar transmitter and the radar receiver; the radar receiver uses a quadrature down-conversion structure, and is connected to an excitation signal source through a filter, a mixer and a digital-to-analog (DA) converter; the mixer is connected to the radar transmitter through a local oscillator and an amplifier in sequence; the radar transmitter emits an electromagnetic wave to a moving object to be measured, and the electromagnetic wave is reflected by a surface of the moving object and received by the radar receiver.

The radar transmitter and the radar receiver are integrated in the same radio frequency (RF) frontend module.

The DC offset compensation specifically includes:
drawing, based on a constellation diagram of two quadrature signals I and Q output by a baseband of the radar receiver during an actual measurement process of a moving object in the presence of an interfering signal, a circle or an arc curve with a DC offset (DC_{I}(t),DC_{Q}(t)) as a center and an amplitude A_{R}(t) as a radius curve:

$${[ I ( t ) - {DC}_{I} ( t ) ]}^{2} + {[ Q ( t ) - {DC}_{Q} ( t ) ]}^{2} = {A}_{R}^{2} ( t )$$

wherein I and Q denote two quadrature signals, and DC_{I}(t) and DC_{Q}(t) denote DC offset components of the quadrature signal I and Q, respectively.

A measurement process of a moving object in the presence of an interfering signal includes: randomly selecting three sampling points from all sampling points of the baseband signal output by the radar receiver to define a circle and a center thereof; calculating a distance from all the sampling points to the center of the circle, and comparing the distance with a set distance threshold; outputting, if a proportion of sampling points with a distance less than the set distance threshold to all the sampling points is greater than a proportion threshold (90% in a specific implementation), the circle and the center thereof; randomly re-selecting, if the proportion of the sampling points with a distance less than the set distance threshold to all the sampling points is not greater than the proportion threshold, three sampling points to define a circle and a center thereof, until a satisfactory center is found, that is, the proportion of sampling points with a distance less than the set distance threshold to all the sampling points is greater than the proportion threshold, or a number of random re-selections exceeds a set value; selecting, if no satisfactory center is found when the number of random re-selections exceeds the set value, a previously defined circle and a center of the circle which correspond to a highest proportion, and taking an abscissa and an ordinate of the center of the circle as DC offset components of two quadrature signals I and Q, respectively, the two quadrature signals being output by the baseband; and subtracting the coordinates of the center of the circle from the two quadrature signals respectively, that is, subtracting the corresponding DC offset components from the quadrature signals I and Q, respectively, so as to realize correction and compensation of the quadrature signals I and Q.

The above compensation method can simply and effectively derive and eliminate the DC offset components in the presence of various interfering signals.

Human cardiac motion causes a displacement of the thoracic skin surface. The amplitude of a displacement caused by breathing is between 10 mm and 20 mm, and the amplitude of a displacement caused by heartbeat is between 0.2 mm and 0.5 mm. The present disclosure detects the heartbeat-induced slight displacement through a Doppler radar sensor with high linearity and high sensitivity.

The present disclosure linearly extracts the displacement information from the signal scattered by the skin and acquires a human cardiogram, referred to as a Doppler cardiogram (DCG). The DCG displays corresponding information of a conventional ECG, and also offers real-time volume, velocity and acceleration information during atrial and ventricular systole and diastole that cannot be observed in the conventional ECG, which are desirable for hospitalization and daily care.

The present disclosure has the following beneficial effects:

Compared with traditional computed tomography (CT) and NMR techniques, the present disclosure features low cost and low radiation hazard. Compared with other contact cardiogram measurements, the present disclosure avoids direct contact with the human body, and provides a convenient and easy way for daily cardiac diagnostic measurements for special patients and in special occasions.

### BRIEF DESCRIPTION OF THE DRAWINGS

FIG. 1A and FIG. 1B show changes in atrial and ventricular volumes, measured by MRI, of a healthy person and a patient with congenital heart disease, and curves and characteristic points of resulting slight displacements of skin surfaces;

FIG. 2 is a specific radar sensor system used in the present disclosure; and

FIG. 3 is a cardiogram, including curves of displacement, velocity and acceleration, measured by a method of the present disclosure, and an ECG measured at the same time.

### DETAILED DESCRIPTION OF THE EMBODIMENTS

An implementation process of the present disclosure will be described in detail below with reference to the drawings in the embodiments of the present disclosure.

As shown in FIG. 2, in a specific implementation, a radar sensor system includes a radar transmitter and a radar receiver. The radar receiver is connected to an excitation signal source through a filter, a multiplier and a DA converter in sequence. The multiplier is connected to the radar transmitter through a crystal oscillator and an amplifier in sequence. The radar transmitter emits an electromagnetic wave to a human thoracic/dorsal skin surface, and the electromagnetic wave is reflected and received by the radar receiver. The radar transmitter and the radar receiver are integrated in the same RF frontend module.

In the specific implementation, the radar sensor is used for measurement. The radar transmitter emits an electromagnetic wave to a stationary dorsal surface to be measured, and the radar receiver receives a reflected wave modulated by the simultaneous atrial and ventricular motion, performs down-conversion and baseband signal demodulation processing, and acquires a baseband signal including cardiac motion information.

The down-conversion and baseband signal demodulation processing are as follows: The baseband signal generated by the reflected wave after down-conversion processing is B(t):

$$B ( t ) = A ( t ) cos [ {θ}_{0} + 4 π \frac{x ( t )}{λ} + {φ}_{0} ( t ) ]$$

wherein A(t) is a signal amplitude; θ₀ is a fixed phase shift, which is related to an initial distance between the RF frontend module and the object to be measured; x(t) is a displacement curve including cardiac motion information; Δ is a carrier wavelength; φ₀(t) is a residual phase noise, which is ignorable.

A baseband signal x(t) including cardiac motion information is acquired through down-conversion, AD conversion and baseband demodulation.

As shown in FIG. 2, the specific working process and working principle of the present disclosure are as follows:

The radar sensor emits an electromagnetic wave signal to the human dorsal skin surface, and the electromagnetic wave signal is reflected by the human dorsal skin surface. The RF frontend module receives the reflected electromagnetic wave signal, and performs quadrature down-conversion on the reflected electromagnetic wave signal to generate two signals Q and I, which are expressed as follows:

$$I ( t ) = {A}_{I} ( t ) cos [ θ + \frac{4 π x ( t )}{λ} ] + {DC}_{I} ( t ) \\ Q ( t ) = {A}_{Q} ( t ) sin [ θ + \frac{4 π x ( t )}{λ} ] + {DC}_{Q} ( t )$$

wherein A_{I}(t) and A_{Q}(t) are respectively the amplitudes of the signals I and Q, the imbalance of which is ignorable due to the quadrature demodulation in the digital domain, that is, A(t)=A_{Q}(t)=A_{R}(t); θ is a fixed phase shift, which is related to an initial distance between the RF frontend module and the object to be measured; x(t) is displacement information of the moving object;λ is a carrier wavelength; and DC_{I}(t) and DC_{Q}(t) are DC offsets of the signals I and Q, respectively.

(2) In the measurement of heartbeat-induced slight motion, the DC offset changes in the signals Q and I are small, and the amplitude changes are also small, which is considered unchanged. Therefore, the signals I and Q form an arc curve with the DC offset (DC_{I}(t),DC_{Q}(t)) as a center and the amplitude A_{R}(t) as a radius.

$${[ I ( t ) - {DC}_{I} ( t ) ]}^{2} + {[ Q ( t ) - {DC}_{Q} ( t ) ]}^{2} = {A}_{R}^{2} ( t )$$

In order to eliminate the influence of the changing DC offset signal on the final imaging result, a DC offset compensation algorithm is used to correct and compensate the signals Q and I.

(3) The signals Q and I are processed by an arctangent algorithm to obtain the phase information:

$$x ( t ) = \frac{λ}{4 π} \{ arctan \frac{[ Q ( t ) - {DC}_{Q} ( t ) ] / {A}_{Q} ( t )}{[ I ( t ) - {DC}_{I} ( t ) ] / {A}_{I} ( t )} - θ \}$$

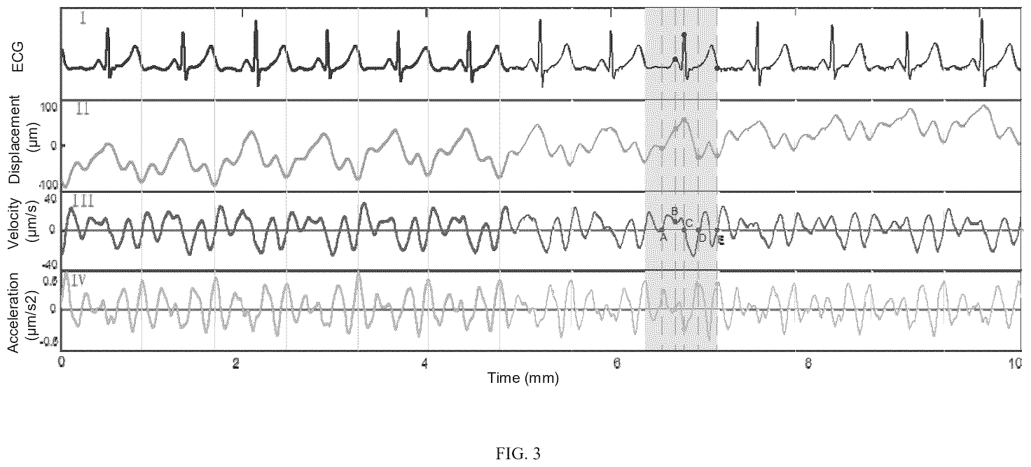

(4) FIG. 3 shows a cardiogram measured using the system. It offers a curve of a heartbeat-induced thoracic displacement, and also offers velocity and acceleration information acquired by derivation of the displacement curve. Taking a cardiac cycle marked in gray as an example, a corresponding relationship between the cardiac curve and an ECG measured by an electrocardiograph at the same time is observed. The relationship is a correspondence between key points determined according to inflection points, concave points, zero points and extreme points on the curves of displacement, velocity and acceleration and those in the ECG. Specifically, the point B corresponds to a P wave, the point C corresponds to a QRS wave, and the point E corresponds to the end of a T wave.

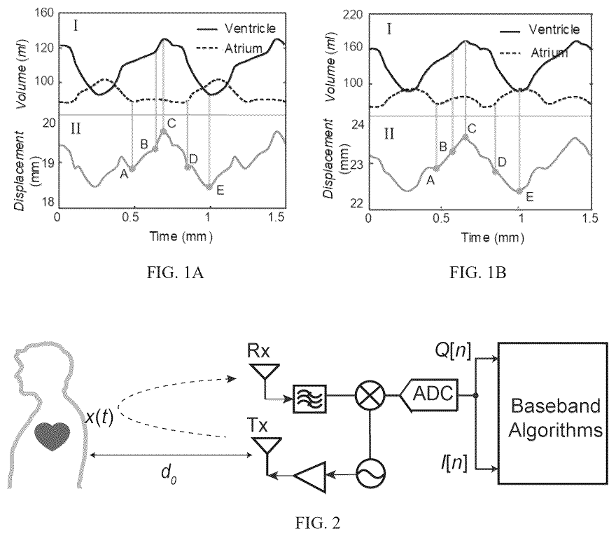

FIG. 1A and FIG. 1B show variation curves of heart volumes of a healthy person and a patient with congenital heart disease, measured by NMR in two cycles. These curves are used for medical research, and yield the heartbeat-induced displacement curves. The displacement curves display the characteristic points A to E corresponding to different stages of atrial and ventricular systole and diastole, which are marked with black dots in FIG. 1A and FIG. 1B.

By comparison, the cardiogram measured by the method of the present disclosure conforms to the actual motion trend of the medical measurement in FIG. 1A and FIG. 1B, which proves the effectiveness of the system.

The above described are merely specific implementations of the present disclosure, and the protection scope of the present disclosure is not limited thereto. Any modification or replacement easily conceived by those skilled in the art within the technical scope of the present disclosure should fall within the protection scope of the present disclosure. Therefore, the protection scope of the present disclosure should be subject to the protection scope defined by the claims.

What is claimed is:

1. A non-contact cardiogram measurement method, comprising: measuring, in a non-contact manner, a slight displacement of a dorsal skin surface induced by changes in atrial and ventricular volumes during a combined atrial and ventricular motion, and acquiring a cardiogram depicting the atrial and ventricular motion in a cardiac cycle, wherein
a measurement is performed from a back of a subject, and a radar sensor is placed facing a dorsal skin of the subject;
the method comprises: measuring, by a Doppler radar or a pulse radar, a distance change between the Doppler radar or the pulse radar and a skin surface through an electromagnetic wave, a light wave and a sound wave; deriving a displacement curve indicating a change in a human heart volume; taking a first-order derivation and a second-order derivation of the displacement curve to acquire a velocity curve and an acceleration curve; taking zero points and extreme points of the velocity curve and the acceleration curve as velocity and acceleration information, and as characteristic points of different stages of atrial and ventricular systole and diastole; and acquiring time, volume change, velocity and acceleration information of atrial and ventricular systole and diastole in the cardiac cycle; and
the radar sensor is used to measure a skin surface displacement, and a direct current (DC) offset compensation is performed during a measurement process, comprising: locating a center of a circle of a baseband signal constellation diagram based on a baseband signal acquired by g quadrature down-conversion of a radar receiver in the radar sensor, determining a DC offset component by a center coordinate, and correcting and compensating the baseband signal by the DC offset component.

2. The non-contact cardiogram measurement method according to claim 1, wherein the radar sensor comprises a radar transmitter and the radar receiver; the radar receiver uses a quadrature down-conversion structure, and is connected to an excitation signal source through a filter, a mixer and a digital-to-analog (DA) converter; the mixer is connected to the radar transmitter through a local oscillator and an amplifier in sequence; and the radar transmitter emits the electromagnetic wave to a moving object to be measured, and the electromagnetic wave is reflected by a surface of the moving object and received by the radar receiver.

3. The non-contact cardiogram measurement method according to claim 1, wherein a measurement process of a moving object in the presence of an interfering signal comprises: randomly selecting three sampling points from all sampling points of the baseband signal output by the radar receiver to define a circle and a center of the circle; calculating a distance from all the sampling points to the center of the circle, and comparing the distance with a set distance threshold; outputting the circle and the center of the circle if a proportion of sampling points with the distance less than the set distance threshold to all the sampling points is greater than a proportion threshold; randomly re-selecting, if the proportion of the sampling points with the distance less than the set distance threshold to all the sampling points is not greater than the proportion threshold, three sampling points to define a circle and a center of the circle, until a satisfactory center of circle is found, that is, the proportion of sampling points with the distance less than the set distance threshold to all the sampling points is greater than the proportion threshold, or a number of random re-selections exceeds a set value; selecting, if no satisfactory center of circle is found when the number of random re-selections exceeds the set value, a previously defined circle and a center of the circle corresponding to a highest proportion; taking an abscissa and an ordinate of the center of the circle as the DC offset component of a quadrature signal I and the DC offset component of a quadrature signal Q, respectively, the two quadrature signals being output by a baseband; and subtracting the coordinates of the center of the circle from the two quadrature signals, respectively, that is, subtracting the corresponding DC offset components from the quadrature signals I and Q, respectively, so as to realize correction and compensation of the quadrature signals I and Q.
